(19) United States
(12) Patent Application Publication
(10) Pub. No.: US 2011/0182911 A1
(43) Pub. Date: Jul. 28, 2011

(54) USE OF IMMOBILIZED ANTAGONISTS FOR ENHANCING GROWTH FACTOR CONTAINING BIOIMPLANT EFFECTIVENESS

(75) Inventors: Cameron M. L. CLOKIE, Toronto (CA); Sean A. F. PEEL, Oakville (CA)

(73) Assignee: INDUCE BIOLOGICS INC., Toronto (CA)

(21) Appl. No.: 13/002,444

(22) Filed: Jan. 3, 2011

Related U.S. Application Data
(63) Continuation of application No. PCT/CA2009/000883, filed on Jul. 3, 2009.
(60) Provisional application No. 61/078,181, filed on Jul. 3, 2008.

(30) Foreign Application Priority Data
Feb. 12, 2009 (CA) 2653866

(51) Int. Cl. A61K 39/395; A61K 38/18; A61K 38/30; A61P 43/00

(52) U.S. Cl. 424/158.1; 514/7.6; 514/8.9; 514/8.8; 514/8.5; 514/8.6

(57) ABSTRACT

A method of retaining growth factors on a substrate for release at a treatment region comprises immobilizing an antagonist of the growth factor on a substrate and binding of the growth factor to the antagonist. In one aspect, the substrate is provided on a bioimplant. The resulting bioimplant allows for activity of the growth factor to continue at the region of implantation. According to the method of the invention, exogenous growth factors can be used to stimulate the repair of various tissues and organs at the site requiring repair, and be protected from inactivation, sequestration or degradation. The invention also provides bioimplants and methods of delivering growth factors.

### CROSS REFERENCE TO RELATED APPLICATIONS

The present application is a National Entry of PCT application number PCT/CA2009/000883, filed Jul. 3, 2009, which claims priority from U.S. patent application No. 61/078,181, filed Jul. 3, 2008 and Canadian patent application number 2,653,866, filed Feb. 12, 2009. The entire contents of such prior applications are incorporated herein by reference.

### FIELD OF THE INVENTION

This invention relates to the field of bioimplant material, and in particular to a method of enhancing the retention and increasing the effectiveness of growth factors associated with a bioimplant using immobilized growth factor binding antagonists.

### BACKGROUND OF THE INVENTION

Growth factors (GFs) are peptides and proteins that stimulate the growth and/or differentiation of cells via the interaction of the GFs with specific cell surface receptors. Growth factors play an integral role in the repair and regeneration of tissues and exogenous GFs can be used to stimulate the repair of various tissues and organs including bone, cartilage, skin and mucosa and to enhance repair through the stimulation of angiogenesis at the repair site.

The transforming growth factor beta (TGFβ) superfamily of secreted growth and differentiation factors in mammals has over 30 members. These dimeric proteins are characterized by a strongly conserved cystine knot-based structure. They regulate the proliferation, differentiation and migration of many cell types, and therefore have important roles in morphogenesis, organogenesis, tissue maintenance and wound healing. The TGFβ superfamily of growth factors can be subdivided into several subfamilies including the transforming growth factor beta family, the bone morphogenetic protein (BMP) and growth and differentiation factor (GDF) family (also called the BMP subfamily), and the inhibin and activin subfamily.

The BMP subfamily of the TGFβ superfamily comprises at least fifteen proteins, including BMP-2, BMP-3 (also known as osteogenin), BMP-3b (also known as growth and differentiation factor 10, GDF-10), BMP-4, BMP-5, BMP-6, BMP-7 (also known as osteogenic protein-1, OP-1), BMP-8 (also known as osteogenic protein-2, OP-2), BMP-9, BMP-10, BMP-11 (also known as growth and differentiation factor 8, GDF-8, or myostatin), BMP-12 (also known as growth and differentiation factor 7, GDF-7), BMP-13 (also known as growth and differentiation factor 6, GDF-6), BMP-14 (also known as growth and differentiation factor 5, GDF-5), and BMP-15 (for a review, see e.g., Azari et al. Expert Opin Invest Drugs 2001;10:1677-1686).

BMPs have been shown to stimulate matrix synthesis in chondroblasts; stimulate alkaline phosphatase activity and collagen synthesis in osteoblasts, induce the differentiation of early mesenchymal progenitors into osteogenic cells (osteoinductive), regulate chemotaxis of monocytes, and regulate the differentiation of neural cells (for a review, see e.g., Azari et al. Expert Opin Invest Drugs 2001;10:1677-1686 and Hoffman et al. Appl Microbiol Biotech 2001;57:294-308).

One of the many functions of BMP proteins is to induce cartilage, bone, and connective tissue formation in vertebrates. The most osteoinductive members of the BMP subfamily are BMP-2, BMP-4, BMP-6, BMP-7, BMP-8 and BMP-9, (see, e.g., Hoffman et al. Appl Microbiol Biotech 2001;57-294-308, Yeh et al. J Cellular Biochem. 2005; 95-173-188 and Boden. Orthopaedic Nursing 2005;24:49-52). This osteoinductive capacity of BMPs has long been considered very promising for a variety of therapeutic and clinical applications, including fracture repair; spine fusion; treatment of skeletal diseases, regeneration of skull, mandibular, and bone defects; and in oral and dental applications such as dentogenesis and cementogenesis during regeneration of periodontal wounds, bone graft, and sinus augmentation. Currently, recombinant human BMP-2 sold as InFUSE™ by Medtronic and recombinant human BMP-7 sold as OP-1® by Stryker are FDA approved for use in spinal fusion surgery, for repair of fracture non-unions and for use in oral surgery.

Other therapeutic and clinical applications for which BMPs are being developed include; Parkinson's and other neurodegenerative diseases, stroke, head injury, cerebral ischemia, liver regeneration, acute and chronic renal injury (see, e.g., Azari et al. Expert Opin Invest Drugs 2001;10:1677-1686; Hoffman et al. Appl Microbiol Biotech 2001;57:294-308; Kopp Kidney Int 2002;61:351-352; and Boden. Orthopaedic Nursing 2005;24:49-52). BMPs also have potential as veterinary therapeutics and as research or diagnostic reagents (Urist et al. Prog Clin Biol Res. 1985;187:77-96).

Three members of the transforming growth factor beta subfamily (TGFβ-1, -2, -3) of the TGFβ superfamily exist in mammals. The TGFβs are highly pleiotropic cytokines that play important roles in wound healing, angiogenesis, immunoregulation and cancer. Therapeutically exogenous TGFβ has been used to promote bone and cartilage repair, wound repair and angiogenesis.

The insulin-like growth factors (IGFs) are a family of 2 growth factors, IGF-1 and IGF-2 with high sequence similarity to insulin. IGF-I has been reported to exert a wide range of biological activities including stimulation of cell proliferation, differentiation and migration, protection from protein degradation and apoptosis, as well as regulation of endocrine factors such as growth hormone. IGF-II has similar properties to IGF-I but appears to be more relevant to carcinogenesis and fetal and embryonic development, IGF-I having a greater role in postnatal development. Therapeutically, recombinant human IGFs (rhIGFs) have been used to promote bone repair, and wound healing.

Other recombinant growth factors that have been used exogenously to enhance tissue repair include members of the fibroblast growth factor superfamily (FGFs), members of the platelet derived growth factor superfamily (PDGFs), epidermal growth factor (EGF) and vascular endothelial growth factor (VEGF).

However for these growth factors to be effective they must be retained at the repair site at a sufficient concentration and at the time when the appropriate responsive cells are present. The short half-life, thermal instability, sensitivity to proteases and/or solubility of the GFs require their administration in combination with a carrier to achieve this requirement.

A number of carriers have been evaluated for the delivery of GFs. These include fibrous collagen sponges, gelatin hydrogels, fibrin gels, heparin, reverse phase polymers such as the poloxamers, scaffolds composed of poly-lactic acid (PLA), poly-glycolic acid (PGA) or their co-polymers (PLGA), heparin-conjugated PLGA scaffolds, porous calcium phosphate cement and a porous hydroxyapatite composite. However, these carriers are of limited effectiveness, due to poor retention of the GF at the implantation site, and poor protection from proteolysis and degradation. Thus the growth factors must be delivered at physiologically high doses to have an effect. This may cause adverse effects and result in high treatment cost.

For example, the current commercial rhBMP-2 containing bioimplant, available under the name Infuse® (Medtronic), uses a type I collagen sponge as the carrier. However over 90% of the BMP-2 is released from the implant within 24 hours of incubation with buffer, long before BMP responsive cells would be expected to have migrated into the implantation site.

Therefore, a need exists in the art for materials and methods for the improved localized delivery and retention of biologically active GFs at the required site over the time period required. A local delivery system may be especially important for human applications, where proportionately higher doses of GFs are required than compared to smaller animals.

One strategy is to chemically immobilize the GF directly onto the carrier retain and it at the implant site. However this can result in partial or complete loss of activity of the GF, and restricts the GF activity such that only those cells directly in contact with the carrier are able to interact with the GF and respond.

Another strategy is to make the carrier of a GF binding material. While collagen has been used as a BMP carrier due to the binding of BMP to collagen, the strength of interaction has been insufficient to retain most of the BMPs within the collagen matrices.

The actions of most growth factors are tightly regulated in the body by the presence of GF binding proteins which are generally antagonists (inhibit the activity of the growth factor). Many of these binding proteins bind to the growth factor with a high affinity. For example Noggin has been reported to bind to rhBMP-2 with a KD of 3×10⁻¹⁰M (Piccolo et al. Cell 1996, 86:589-98) and can completely inhibit BMP activity at a 1:1 molar ratio (see Example 1). However because of the inhibitory nature of these antagonists on GF activity they have not been considered useful as a growth factor carrier.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

In one aspect, the invention provides a method of binding an exogenous growth factor to a substrate, the method comprising immobilizing an antagonist of the growth factor on the substrate and binding the growth factor to the antagonist.

In another aspect, the invention provide a method of delivering growth factor activity at a localized site, the method comprising immobilizing an antagonist of the growth factor on a substrate, binding the growth factor to the antagonist, and implanting said substrate at or near the site.

In another aspect, the invention provides a method for adapting a substrate to deliver growth factor activity at a localized site, the method comprising immobilizing an antagonist of the growth factor on the substrate and binding the growth factor to the antagonist.

In another aspect, the invention provides a bioimplant adapted to deliver an exogenous growth factor at a site of implantation, the bioimplant comprising a surface having immobilized thereon an antagonist of the growth factor and wherein the growth factor is bound to the antagonist.

In another aspect, the invention provides a method of delivering growth factor activity to a desired location in an organism comprising: providing a substrate; immobilizing an antagonist of said growth factor on said substrate; binding the growth factor to the antagonist; and, implanting the substrate at the desired location.

In one aspect, the present invention provides methods and materials for enhancing the effectiveness of growth factor containing bioimplants. In accordance with one aspect of the present invention there is provided a method for enhancing the retention of an exogenous growth factor on or in a material comprising the step of immobilizing an antagonist onto a carrier component of the material, wherein said retention is enhanced in comparison to retention observed when said antagonist is not immobilized onto said carrier component.

In accordance with a further aspect of the present invention there is provided a method of enhancing the effectiveness of a growth factor-containing bioimplant by combining the growth factor with an antagonist of the growth factor, wherein the antagonist is immobilized on a carrier.

In accordance with another aspect of the present invention, there is provided a bioimplant comprising a growth factor and an antagonist of said growth factor immobilized on a carrier.

In one aspect, the growth factor (GF) is a member of the transforming growth factor beta superfamily. In particularly preferred embodiments the growth factor is a BMP. In one aspect, the antagonist is a strong antagonist. In one aspect where the GF is a BMP, the antagonist is Noggin.

In one aspect, the growth factor is releasably bound to the antagonist or retains its activity when bound to the antagonist.

In one aspect, the antagonist is an antibody having a binding region that is complementary to a region of the growth factor. The term “region” is meant to indicate an amino acid sequence.

Another aspect of the present invention provides a method of preparing a bioimplant material comprising the step of immobilizing the growth factor antagonist to the carrier.

In one aspect the antagonist is immobilized by lyophilization onto the carrier.

In another aspect the antagonist is immobilized chemically onto the carrier.

In another aspect the antagonist is genetically modified to include an amino acid sequence which promotes binding to the carrier.

In another aspect, the amino acid sequence is a collagen binding sequence.

In another aspect, the carrier is granular. In one aspect, the granular carrier is a calcium phosphate, more particularly hydroxyapatite, beta-tricalcium phosphate or a biphasic calcium phosphate.

In another aspect, the granular carrier is a bioglass.

In another aspect, the granular carrier is type I collagen granules.

A further aspect of the present invention provides a method of producing a gel or putty by combining the carrier-growth factor combination with a delivery vehicle.

In one aspect, the delivery vehicle is a reverse phase polymer. In another aspect, the reverse phase polymer is a poloxamer, more particularly poloxamer 407 (also called Pluronic™ F127).

### BRIEF DESCRIPTION OF THE DRAWINGS

FIG. 1 depicts a graph demonstrating the effect of Noggin on BMP activity in vitro.

FIG. 2 depicts a graph demonstrating the effect of fetuin on BMP activity in vitro

FIG. 3 depicts a graph demonstrating the release of BMP from surfaces coated with Noggin and Fetuin, collagen, albumin or uncoated.

FIG. 4 depicts a graph demonstrating the retention of BMP activity by surfaces coated with Noggin, Fetuin collagen, bovine serum albumin or left uncoated.

FIG. 5 depicts a microCT™ image of BMP induced bone ossicle formed in the in vivo mouse muscle pouch BMP assay.

FIG. 6 depicts 4 graph comparing the 4 quantity measures of the amount of bone produced by implanting BMP-2 and BMP-7 containing bioimplants.

FIG. 7 is a panoramic picture of the bilateral calvarial defects created by merging sectional photomicrographs from Algisorb™+BMP+Pluronic™ versus Algisorb™+BM filled defects at 6 weeks (Group 5). H&E staining. Original magnification, 4×.

FIG. 8 depicts a low power photomicrograph of Algisorb™+BMP+Pluronic™ filled defect at 6 weeks. Large amounts of bone marrow are also present. H&E staining. Original magnification, 10×.

FIG. 9 depicts antagonism of BMP induced ALP increase by pre-incubation with increasing concentrations of antibody.

FIG. 10 depicts retention of BMP activity by surfaces coated with an antiBMP-2 antibody.

### DETAILED DESCRIPTION OF THE INVENTION

Growth factors (GF) play an integral role in the repair and regeneration of tissues and exogenous GFs can be used to stimulate the repair of various tissues and organs. For exogenous growth factors to be effective in stimulating repair they must be retained at the site requiring repair, and be protected from inactivation, sequestration or degradation.

The present invention is based on the unexpected and surprising discovery that immobilized growth factor binding antagonists can be used to retain GFs at a wound site and enhance their biological effectiveness. In a broad sense, the invention comprises the immobilization of such GF antagonist on a surface of an implant and the binding of the GF to the antagonist. The invention also encompasses bioimplants having at least one surface comprising such immobilized antagonists and associated GFs. The invention also provides methods of delivering GF activity to an implant site.

In solution, antagonists of the invention would normally inhibit or block the activity of the respective GF. As such, the findings described herein provide a novel means of enhancing the activity of GFs at specific sites. The present inventors have developed methods and materials for enhancing the efficacy of bioimplants by improving the retention of growth factors at sites of implantation, while maintaining the growth factor activity. In particular, as described further herein, the present inventors have found that binding growth factors to antagonists that are immobilized on a substrate results in an effective localized activity of such growth factors. In one aspect, the methods and materials make use of antagonists for growth factors, wherein the antagonists are immobilized on a carrier.

In another aspect, the bioimplant of the present invention comprises a growth factor bound to an antagonist of the growth factor that immobilized on a surface on the bioimplant. The bioimplant can be used for a variety of therapeutic and/or clinical applications, including fracture repair; bone grafts; spine fusion; regeneration of skull, mandibular, and bone defects; oral and dental applications such as dentogenesis and cementogenesis during regeneration of periodontal wounds, bone graft, and sinus augmentation; dermal and ulcer repair and bladder wall repair.

It will be understood that for the immobilization of the antagonists, the surface of the implant may be treated to enhance or otherwise permit such immobilization. As discussed herein, this may be achieved by using a carrier material that serves to bind the antagonists to the surface of the implant. Although this is preferred, it will be understood that any means of adhering the antagonists of the invention to a surface can be used. To further assist the immobilization, it will be understood that the surface to which the antagonist are adhered may be provided with a physical texture or treatment that will enhance binding of the antagonists/carriers.

The present inventors have studied the ability of various proteins to retain BMP and their effect on BMP activity (Clokie et al., The Effect of Non-Collagenous Proteins on BMP Retention and Activity (abstract), Univ. of Toronto Faculty of Dentistry Research Day Book of Abstracts, Feb. 12, 2008). The entire contents of such abstract and related poster presentation are incorporated herein by reference.

As will be understood by the present disclosure, the bioimplant of the invention can be used for delivering growth factor activity to any desired location in a body.

Definitions

Unless defined otherwise, all technical and scientific terms used herein have the same meaning as commonly understood by one of ordinary skill in the art to which this invention belongs.

As used herein the term “bioimplant” or “implant” refers to a material which is suitable for implantation. In one aspect of the invention, and as described further herein, at least one surface of the bioimplant is provided with an exogenous growth or biologically active factor having a therapeutic and/or clinical activity or effect.

The term “substrate” as used herein refers to a surface that is adapted to have immobilized thereon the antagonists of the invention. In one aspect, the substrate comprises a surface of a bioimplant. In another aspect, the substrate may comprise or include a coating, carrier, surface treatment etc. that serves to assist in immobilizing the antagonists.

As used herein the term “growth factor” refers to a peptide or protein that stimulates the growth and/or differentiation of cells via the interaction of the GFs with specific cell surface receptors. Examples of growth factors include the bone morphogenetic proteins (BMPs), transforming growth factor beta (TGFβ), the insulin-like growth factors (IGF), the fibroblast growth factors (FGFs), platelet derived growth factor (PDGF) and vascular endothelial growth factor. In preferred embodiments the growth factors are members of the TGFβ superfamily. In particularly preferred embodiments they are BMPs

As used herein the term “growth factor antagonist” or “antagonist” refers to a molecule which, when in solution, prevents the specific growth factor for which it is an antagonist from stimulating the growth and/or differentiation of a target cell. In one aspect, the antagonist completely inhibits the activity of the growth factor at a molar ratio equal to or less than about 1:100 (GF:antagonist). In another aspect, the antagonist inhibits the GF activity at a molar ratio equal to or less than 1:10 (GF:antagonist).

As used herein in the term “growth factor binding antagonist” or “binding antagonist” refers to an antagonist which exerts its antagonistic effect on the respective growth factor by binding thereto. Examples of growth factor binding antagonists for BMPs include Noggin, Chordin, Dan and Gremlin. Examples of growth factor binding antagonists for TGF-β include the latency associated peptide (LAP). Examples of growth factor binding antagonists for the IGFs are the IGF binding proteins (IGFBPs). Other binding antagonists can be created by producing an activity neutralizing antibody for the growth factor or by a soluble growth factor receptor. An example of such a receptor would be the human transforming growth factor soluble receptor Type II (rhTGFbsRII; see Tsang et al. 1995, Cytokine 7:389)

As used herein the term “strong antagonist” means an antagonist that is capable of inhibiting growth factor activity at molar ratios of 10:1 (antagonist:GF) or less. As an example, a strong BMP antagonist would be Noggin, which completely inhibits rhBMP-2 stimulation of alkaline phosphatase activity in C2C12 cells at a molar ratio of <2:1 (see example 1)

As used herein the term “moderate antagonist” means an antagonist that is capable of inhibiting growth factor activity at a molar ratio less than or equal to 1000:1, but greater than 10:1 (antagonist:GF).

As used herein “weak antagonist” means an antagonist that either cannot completely inhibit the activity of the growth factor or only does so at a molar ratio of greater than 1000:1. As an example, a weak antagonist of rhBMP-4 is fetuin which does not completely inhibit rhBMP-4 stimulation of alkaline phosphatase in C2C12 cells at a molar ratio of 50,000:1 (see example 1).

The term “recombinant” refers to a protein produced by a transiently transfected, stably transfected, or transgenic host cell or animal as directed by an expression construct containing the cDNA for that protein. The term “recombinant” also encompasses pharmaceutically acceptable salts of such a polypeptide

As used herein, the term “polypeptide” or “protein” refers to a polymer of amino acid monomers that are alpha amino acids joined together through amide bonds. Polypeptides are therefore at least two amino acid residues in length, and are usually longer. Generally, the term “peptide” refers to a polypeptide that is only a few amino acid residues in length. A polypeptide, in contrast with a peptide, may comprise any number of amino acid residues. Hence, the term polypeptide includes peptides as well as longer sequences of amino acids.

As used herein, the terms “bone morphogenetic protein” or “bone morphogenic protein” or “BMP” are used interchangeably and refer to any member of the bone morphogenetic protein (BMP) subfamily of the transforming growth factor beta (TGFβ) superfamily of growth and differentiation factors, including BMP-2, BMP-3 (also known as osteogenin), BMP-3b (also known as growth and differentiation factor 10, GDF-10), BMP-4, BMP-5, BMP-6, BMP-7 (also known as osteogenic protein-1, OP-1), BMP-8 (also known as osteogenic protein-2, OP-2), BMP-9, BMP-10, BMP-11 (also known as growth and differentiation factor 8, GDF-8, or myostatin), BMP-12 (also known as growth and differentiation factor 7, GDF-7), BMP-13 (also known as growth and differentiation factor 6, GDF-6), BMP-14 (also known as growth and differentiation factor 5, GDF-5), and BMP-15.

The terms “bone morphogenetic protein”, “bone morphogenic protein” and “BMP” also encompass allelic variants of BMPs, function conservative variants of BMPs, and mutant BMPs that retain BMP activity. The BMP activity of such variants and mutants may be confirmed by any of the methods well known in the art (see the section Assays to characterize BMP, below) or as described in Example 1

In preferred embodiments, the BMP is BMP-2, BMP-4, BMP-5, BMP-6, BMP-7, BMP-8 or BMP-9. In particularly preferred embodiments the BMP is BMP-2, BMP-4 or BMP-7.

In preferred embodiments the BMP is a mammalian BMP (e.g., mammalian BMP-2 or mammalian BMP-7). In particularly preferred embodiments, the BMP is a human BMP (hBMP) (e.g. hBMP-2 or hBMP-7).

As used herein the term “carrier” refers to a material component of a biomaterial, such as bioimplant, whose purpose is to provide a scaffold for new tissue repair and/or to retain the exogenous growth factor within the wound site. In one embodiment the carrier is a synthetic or natural calcium phosphate, such as a hydroxyapatite or a beta-tricalcium phosphate, a mixture of both, or natural bone mineral such as BioOss. In another embodiment the carrier is a synthetic or natural polymer, such as a polyglycolic acid, polylactic acid, a mixture of both, or a chitin. In another embodiment the carrier is a metal, such as titanium its alloys and oxides. In another embodiment the carrier is a bio-active glass, such as Bioglass. In another embodiment the carrier is a protein, such as collagen type I, or fibrin or silk.

As used herein the term “delivery vehicle” refers to a material which when combined with the bioimplant improves its handling properties, such as binding together the carrier granules to form a putty or to make the bioimplant “flowable” permitting its delivery via syringe. In preferred embodiments the delivery vehicle is a reverse phase polymer. In particularly preferred embodiments the reverse phase polymer is a poloxamer, more particularly Pluronic™ F127 (also called poloxamer 407).

Assays to Measure BMP Activity

Assays to characterize in vitro and in vivo function of recombinant BMPs are well known in the art, (see, e.g., U.S. Pat. No. 4,761,471; U.S. Pat. No. 4,789,732;.U.S. Pat. No. 4,795,804; U.S. Pat. No. 4,877,864; U.S. Pat. No. 5,013,649; U.S. Pat. No. 5,166,058; U. S. Patent No. 5,618,924; U.S. Pat. No. 5,631,142; U.S. Pat. No. 6,150,328; U.S. Pat. No. 6,593,109; Clokie and Urist Plast. Reconstr. Surg. 2000;105:628-637; Kirsch et al. EMBO J 2000;19:3314-3324; Vallejo et al. J Biotech 2002;94:185-194; Peel et al. J Craniofacial Surg. 2003;14:284-291; and Hu et al. Growth Factors 2004;22:29-33;

Such assays include: in vivo assays to quantify the osteoinductive activity of a BMP following implantation (e.g., into hindquarter muscle or thoracic area) into a rodent (e.g. a rat or a mouse) (see, for example, U.S. Pat. No. 4,761,471; U.S. Pat. No. 4,789,732; U.S. Pat. No. 4,795,804; U.S. Pat. No. 4,877,864; U.S. Pat. No. 5,013,649; U.S. Pat. No. 5,166,058; U.S. Pat. No. 5,618,924; U.S. Pat. No. 5,631,142; U.S. Pat. No. 6,150,328; U.S. Pat. No. 6,503,109; Kawai and Urist., Clin Orthop Relat Res, 1988;222:262-267; Clokie and Urist, Plast. Reconstr. Surg., 2000;105:628-637; and Hu et al,. Growth Factors 2004;22:29-33); in vivo assays to quantify the activity of a BMP to regenerate skull trephine defects in mammals (e.g., rats, dogs, or monkeys) (see, for example, U.S. Pat. No. 4,761,471 and U.S. Pat. No. 4,789,732); in vitro assays to quantify the activity of a BMP to induce proliferation of in vitro cultured cartilage cells (see, for example, U.S. Pat. No. 4,795,804); in vitro assays to quantify the activity of a BMP to induce alkaline phosphatase activity in in vitro cultured muscle cells (e.g., C2C12 cells (ATCC Number CRL-1772)) or bone marrow stromal cells (e.g., murine W-20 cells (ATCC Number CRL-2623)) (see for example, U.S. Pat. No. 6,593,109; Ruppert et al. Eur J Biochem 1996;237:295-302; Kirsch et al. EMBO J 2000;19:3314-3324; Vallejo et al. J Biotech 2002;94:185-194; Peel et al. J Craniofacial Surg. 2003;14:284-291; and Hu et al. Growth Factors 2004;22:29-33); in vitro assays to quantify the activity of a BMP to induce FGF-receptor 2 (FGFR3) expression in cultured mesenchymal progenitor cell lines (e.g., murine C3H10T1-2 cells) (see, for example, Vallejo et al. J Biotech 2002;94:185-194); in vitro assays to quantify the activity of a BMP to induce proteoglycan synthesis in chicken limb bud cells (see, for example, Ruppert et al. Eur J Biochem 1996;237:295-302); and in vitro assays to quantify the activity of a BMP to induce osteocalcin treatment in bone marrow stromal cells (e.g., murine W-20 cells (ATCC Number CRL-2623)) (see, for example, U.S. Pat. No. 6,593,109).

Assays to Identify BMP Antagonists

Various assays can be used to determine whether a substance is a BMP antagonist. For example using one of the BMP activity assays described above the BMP can first be co-incubated with the antagonist at different molar ratios before being tested in the assay. If the substance is an antagonist the effect of the BMP in the assay will be reduced compared to the effect of BMP alone. It is also possible to evaluate whether the antagonists are strong, moderate or weak antagonists by determining the molar ratio of antagonist required to completely inhibit the effect of the BMP. For example if the molar ratio is equal to or less than 10:1 (antagonist:GF) the antagonist could be considered a strong antagonist, if less than or equal to 1000:1 a moderate antagonist and if more than 1000:1 or the BMP activity could not be completely inhibited a weak antagonist.

Assays to Measure BMP Binding and Release

Various assays can be used to measure binding and release of recombinant BMP from a carrier. For example, the amount of recombinant BMP protein can be quantified by any of the techniques well known in the art, including dot blots, immunoassay (e.g., enzyme linked immunosorbent assays, ELISA), chromatography (e.g., high pressure liquid chromatography, HPLC and ion-exchange chromatography) and surface plasmon resonance (SPR).

Such methods are well known in the art (see, for example, such methods are well known in the art (See for example, Harlow and Lane. Using Antibodies: A Laboratory Manual. Cold Spring Harbor Laboratory Press. 1999; Gosling, ed. Immunoassays: A Practical Approach. Oxford University Press. 2000; Oliver, ed. HPLC of Macromolecules: A Practical Approach. Oxford University Press, 1998; Millner, ed. High Resolution Chromatography: A Practical Approach. Oxford University Press, 1999; Hockfield et al. Selected Methods for Antibody and Nucleic Acid Probes. Cold Spring Harbor Laboratory Press. 1993; Gore, ed. Spectrophotometry and Spectrofluorimetry: A Practical Approach. Oxford University Press, 2000)

For example, protocols for radioimmunoassay analysis of BMP proteins have been described (see, for example, U.S. Pat. No. 4,857,456). For example, protocols for immunoblot analysis of BMP proteins have been described (see, for example, Wang et al. Proc Natl Acad Sci USA 1990;87:2220-2224). For example, ELISA kits for the quantification of protein levels of human, rat, or mouse BMP-2 are commercially available, for example, from R&D Systems (catalog #DBP200, PDBP200, or SBP200). For example, ELISA kits for the quantification of protein levels of human BMP-7 are commercially available, for example, from R&D Systems (catalog #DY354 or DY354E).

### EXAMPLES

The present invention is next described by means of the following examples. However, the use of these and other examples anywhere in the specification is illustrative only, and in no way limits the scope and meaning of the invention or of any exemplified form. Likewise, the invention is not limited to any particular preferred embodiments described herein. Indeed, many modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

### Example 1

### An In Vitro Assay to Evaluate Antagonists of BMPs

An antagonist for a growth factor (GF) can be identified as such by incubating it with the growth factor and then exposing it to GF responsive cells and demonstrating that the effect of the GF is inhibited by the antagonist.

To demonstrate this recombinant human BMPs (rhBMPs) were incubated with different amounts of recombinant mouse Noggin (rmNGN) or bovine fetuin and then the mixture was added to cultures of C2C12 cells to test for BMP activity.

Materials & Methods

Recombinant human BMP-2 (rhBMP-2), rhBMP-4 and recombinant mouse Noggin (rmNoggin) were obtained from RnD Systems (Cat # 355-BM-010/CF, 314-BP-010, 1967-NG). Bovine fetuin (bAHSG) was purchased from Sigma Aldrich (Cat #F6131). Stock solutions were prepared as described by the manufacturers.

In vitro BMP-2 Activity Assay: Alkaline Phosphatase Induction in C2C12 Cells:

The activity of recombinant hBMP proteins was quantified based upon stimulation of alkaline phosphatase activity in cultured C2C12 cells, as has been described (see, for example, Peel et al. J Craniofacial Surg. 2003;14:284-291 and Hu et al. Growth Factors. 2004;22:29033).

C2C12 cells (ATCC accession number CRL-1772, Manassas, Va.) were passaged before confluence and resuspended at 0.5×10⁵ cells/ml in alpha-MEM (Invitrogen) supplemented with 15% heat-inactivated fetal bovine serum, antibiotics and 50 μg/ml ascorbic acid. One ml of cell suspension was seeded per well of a 24 well tissue culture plate (BD Falcon, Fisher Scientific Cat # 08-772-1) and the cells were maintained at 37° C. and 5% CO₂.

After 3 to 24 hours the medium was replaced with 1 ml of fresh media containing a fixed amount (50 or 40 ng/ml) of rhBMP-2 or rhBMP-4 plus Noggin or fetuin at various doses. Controls included cells cultured in media without any test sample or with only Noggin or fetuin. Cultures were maintained for another 1 to 7 days. Medium was changed every two days.

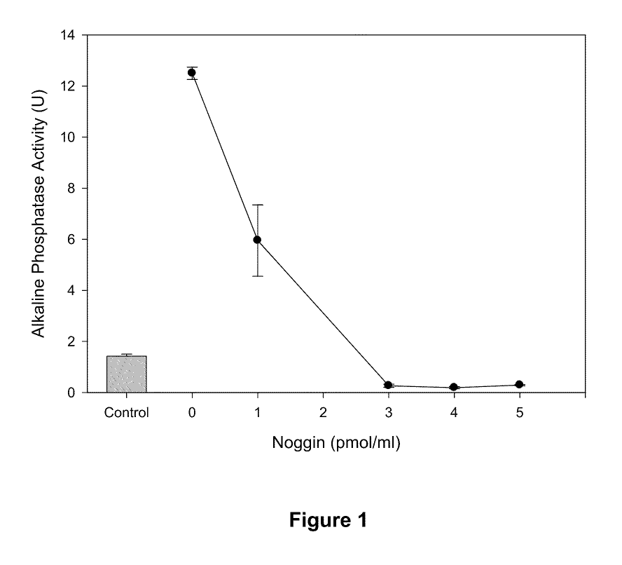

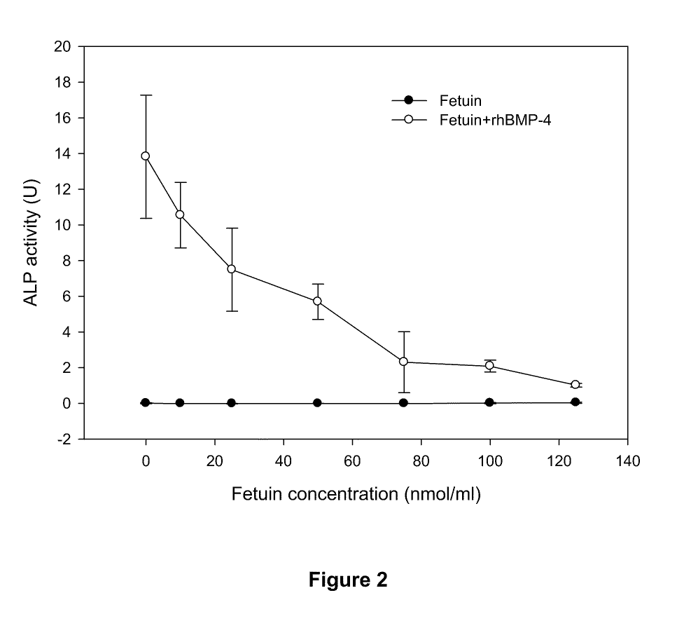

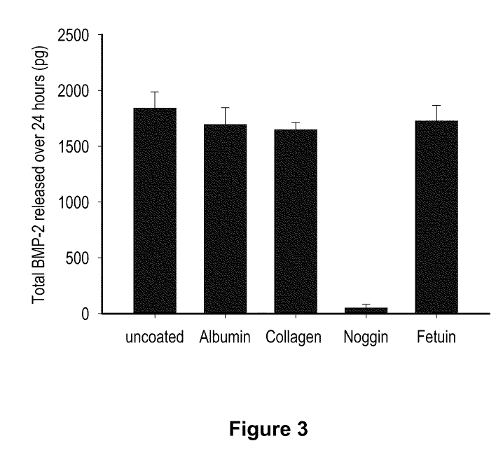

In particular, for the assays summarized in FIG. 1, recombinant mouse Noggin (rmNGN) was incubated with 50 ng/ml (2.7 pmol/m1) of rhBMP-2 and doses of 0, 1, 3, 4 and 5 pmol/ml. The samples were then applied to cultures of C2C12 cells. After 48 hours the cell layers were lysed and the alkaline phosphatase (ALP) activity of the cell layers measured. Similarly, for the assays summarized in FIG. 2, bovine fetuin (bAHSG) was incubated with 40 ng/ml rhBMP-4 (2.2 pmol/ml) at concentrations of 0, 12.5, 25, 50, 75, 100 and 125 nmol/ml. The samples were then applied to cultures of C2C12 cells. After 48 hours the cell layers were lysed and the alkaline phosphatase (ALP) activity of the cell layers measured.

At harvest, the conditioned medium was removed and the cell layers were rinsed with Tris buffered saline (20 mM Tris, 137 mM NaCl, pH 7.4) and M-Per™ lysis buffer (Pierce Biotechnology Inc., Rockford, Ill., catalogue # 78501) was added. The cell layer was scraped into Eppendorf tubes and sonicated. The lysate was centrifuged at 5000 g at 5° C. for 10 minutes, and the supernatant was assayed for alkaline phosphatase (ALP) by monitoring the hydrolysis of nitrophenol phosphate in alkaline buffer (Sigma-Aldrich, St. Louis Mo., catalog P5899) as described in Peel et al. J Craniofacial Surg. 2003;14:284-291 or by using the Alkaline Phosphatase detection kit, Fluorescence (Sigma-Aldrich, catalogue #APF) according to manufacturer's instructions. To normalize the ALP activity the cellular protein content in each well was also assayed using the Coomasie (Bradford) Protein Assay (Pierce Biotechnology Inc., catalogue # 23200). The normalized ALP activity for each sample was calculated by dividing the ALP activity per well by the protein content per well.

The efficacy of the antagonist is assessed by determining the molar ratio of antagonist to BMP at which no significant increase in alkaline phosphatase activity from control can be determined.

Results

As shown in FIG. 1, recombinant mouse Noggin was able to completely inhibit an increase in alkaline phosphatase activity due to treatment with rhBMP-2 in the C2C12 cells at a molar ratio of 1.1:1 (rmNGN:rhBMP-2). In particular, it was found that addition of 50 ng/ml of rhBMP-2 to the culture medium increased ALP activity over six fold in this assay, compared to control cultures receiving culture medium alone. However addition of Noggin to rhBMP-2 containing media at molar ratios of 1.1 to 1 or higher reduced ALP levels to control levels, indicating it is a strong BMP antagonist.

As shown in FIG. 2, bovine fetuin partially inhibited the BMP induced increase in alkaline phosphatase activity but was unable to completely inhibit an increase in alkaline phosphatase due to rhBMP-4 at a molar ratio of 56,820:1 (bAHSG:rhBMP-4). In particular, it was found that addition of 40 ng/ml of rhBMP-4 to the culture medium increased ALP activity over ten fold in this assay, compared to control cultures receiving culture medium alone. However addition of fetuin to rhBMP-4 containing media reduced the ALP activity. However even at molar ratios of 55,000:1 fetuin was unable to completely inhibit BMP stimulation of ALP activity indicating it is a weak antagonist of rhBMP-4.

This example identifies BMP antagonists by their ability to inhibit the BMP stimulated activity in responsive cells. It also demonstrates how the effectiveness of different antagonists can be compared and how to identify the molar ratio at which an antagonist inhibits the respective growth factor.

### Example 2

### An In Vitro Assay for TGFβ Antagonists

In vitro assays for TGFβ activity are well known in the art (see Garrigue-Antar et al. J Immunol Methods. 1995 Oct. 26; 186(2):267-74; Kim et al. Arch Pharm Res Vol 25, No 6, 903-909, 2002, Tesseur et al. BMC Cell Biol. 2006 Mar. 20;7:15).

By using testing mixtures of β and potential antagonists it is possible to evaluate the efficacy of the antagonist has been described in the art (Tsang, M. et al., 1995, Cytokine 7:389) and as described below.

Materials & Methods

Human TGFβ1, -β2 and -β3 and the TGFβ latency associated peptide (LAP) are obtained from RnD Systems (Cat# 240-B-002, 302-B2-010, 243-B3-002, 246-LP) and stock solutions are prepared as recommended by the manufacturer to make 200 μM stock solutions.

Stock cultures of Mv-1-Lu mink lung epithelial cells are obtained from the ATCC (Cat # CCL-64) are grown in alpha MEM supplemented with 10% fetal bovine serum. At the time of assay the cells are subcultured and re-suspended at 1×10⁴ cells/ml and 100 μl of cell suspension are plated into wells of tissue culture treated 96 well plates. After 24 hours a further 100 μl of fresh medium is added containing 0.5 ng/ml of β (final concentration 0.25 ng/ml) and increasing concentrations of LAP. Controls include fresh medium alone and LAP alone at various concentrations.

After a further 48 hours 25 to 50 μl of Alamar™ blue (Invitrogen Cat # DAL 1100) is added to each well. After 1 to 4 hours 200 μl of the medium is transferred to a new 96 well plate and the absorbance of the medium is read at 570 and 600nm. The percentage of the dye reduced is determined according to the manufacturer's directions.

Results

The amount of Alamar™ dye reduced is proportional to the number of cells present in each well. TGFβ reduces the proliferation rate of the Mv-1-Lu cells resulting in a lower percentage of dye reduced. In the presence of LAP the inhibition of proliferation is reduced resulting in a larger cell number and more dye reduced than with TGFβ alone. LAP inhibits TGFβ-1 inhibition of cell proliferation with an ED₅₀ of 25 to 50 ng/ml for 0.25 ng/ml TGFβ-1.

This example identifies TGFβ antagonists and their effectiveness as an antagonist.

### Example 3

### An In Vitro Bioassay for Insulin Like Growth Factor (IGF) Antagonists

Assays for IGF activity are well known in the art (see Van Zoelen, Prog. Growth Factor Res, 1990, 2: 131-152, Karey, K. P. et al., Cancer Research, 1988, 48:4083 and Olebruck et al. Toxicology Let. 1998, 96,97:85-95). These assays can also be used to demonstrate whether a substance is an IGF antagonist as described below.

Materials & Methods

IGF-1, IGF-2, IGFBP-1, -2, -3, -4, -5, -6 and soluble IGF-IIR are obtained from RnD Systems (Cat # 291-G1, 292-G2, 871-B1, 674-B2, 675-B3, 804-GB, 875-B5, 876-B6, 2447-GR).

The MCF-7 cell-line is obtained from the ATCC (cat # HTB-22) and is cultured in DMEM (Invitrogen) containing 5% fetal bovine serum (FBS).

At time of seeding 100 μl solutions of 5,000/cells ml are plated into wells of a 96-well tissue culture plate. After 24 h the medium is changed to serum-free DMEM containing 0.15% bovine serum albumin (BSA). After the medium-change, the cells are not longer able to proliferate and are arrested in G0/G1-part of the cell cycle. By addition of insulin-like growth factor-1 (IGF-1) or insulin-like growth factor-2 (IGF-2) the cells are restimulated to cell division. Other cytokines like epidermal growth factor (EGF), transforming growth factor-beta (TGF-β) or platelet-derived growth factor (PDGF) are not stimulating cell division of this cell line.

At the medium change 200 μl fresh medium (DMEM+0.15% BSA) containing 0.1 to 100 ng/ml IGF plus an IGF antagonist at different molar ratios is added to the wells.

After a culture period of 4 days the cell number was determined using the Alamar™ Blue assay as described above

Results

Addition of IGF-1 at doses of 0.1, 1 and 10 ng/ml significantly increased cell number. Addition of IGF-2 at doses of 10 or 100 ng/ml significantly increase cell number. Addition of IGFBP-1 to IGF-1 inhibits proliferation with an ED₅₀ 1-4 μg/6 ng). Addition of IGFBPs to IGF-2 inhibits proliferation with an ED₅₀ to inhibit 14 ng/ml IGF-2 of: BP-2, 3, 4=50-150 ng/ml; BP-6=100-400 ng/ml; BP-5=5-10 μg/ml.

This example identifies IGF antagonists and their effectiveness as an antagonist.

### Example 4

### An In Vitro Assay for Release of BMPs from Surfaces Coated with BMP Antagonists

To determine whether surfaces coated with various factors can be used to retain the growth factor, various surfaces were coated with various proteins and then incubated the coated surfaces with a solution of BMP that was air dried. The surfaces were then incubated with buffer and the amount of BMP released measured by ELISA.

Materials & Methods

In a first part of this example, wells of 96 well plates were coated with 10 μg/cm² of BSA, collagen, Noggin, fetuin, or left uncoated. 10 ng of rhBMP-2 was added to the wells and air dried. The plates were then incubated with PBS+0.1% BSA. The plates were washed at 0 minutes, 30 minutes, 60 minutes, 4 hours, and 24 hours. The amount of rhBMP-2 released into the buffer at each time interval was measured by a hBMP-2 ELISA

BMP-2 ELISA assay: The amount of BMP-2 released into the buffer was measured using an commercial ELISA (Quantikine hBMP-2 ELISA, RnD Systems Cat # ). The ELISA was carried out according to the manufacturer's instructions.

In the second part of this example, wells of 24 well tissue culture plates were coated with various test proteins by air drying. rhBMP-2 was added to the wells and air dried onto the immobilized coating. Once dry PBS+0.1% BSA buffer was applied to the wells. The buffer was replaced with fresh buffer after 0, 0.5, 1, 4 and 24 hours and the amount of BMP-2 released from each well was assayed by ELISA. The total amount of BMP released over 24 hours was determined and plotted as mean±SD (n=4).

Results

From the first part of this example, it was found that BMP released from uncoated, plates was significantly higher than BMP released by plates coated with the strong BMP antagonist Noggin than the weak BMP antagonist fetuin (P<0.001; ANOVA on Ranks, Tukey post-hoc test). The least BMP released was in the Noggin coated samples (P<0.001).

In the second part of this example, it was found that significantly less BMP was released over 24 hours from the Noggin coated surface than any of the others (P<0.001).

This example demonstrates that a strong antagonist, such as Noggin, retains the growth factor for a longer period on a surface.

### Example 5

### An In Vitro Assay to Test the Activity of BMPs Bound to Surfaces Coated with Antagonists

To demonstrate that the retained growth factor on the coated surface is biologically active, responsive cells can be cultured in contact with the surface and their response to the growth factor measured. Such assays are known in the art (see Peel et al. J. Craniofac. Surg 2003, 14:284-291). As an example surfaces coated with BMP binding antagonists and other proteins were evaluated for retention of BMP activity.

Materials & Methods

Materials were as described in Example 1.

Wells of 24 well tissue culture plates were coated with 10 μg/cm² of the test proteins. The test proteins were bovine serum albumin, type I collagen, bovine fetuin (all from Sigma Aldrich) and recombinant mouse Noggin (RnD Systems). The proteins were prepared in solution as described by the vendor and left to air dry in a laminar flow cabinet.

Cell culture medium, with or without 50 ng rhBMP-2 was added to the plates. In one set of plates the BMP containing medium was removed and replaced with fresh medium without BMP. Myogenic C2C12 cells were seeded onto the plates, cultured for 2 or 5 days, and then assayed for alkaline phosphatase (ALP) activity and protein content as an indicator of osteoinduction.

Results

No significant differences were found in the basal ALP activity of C2C12 cells on the various substrata in the absence of BMP.

ALP activity in cultures grown in the presence of BMP was significantly higher than in those cultured in the absence of BMP in all groups (P<0.001).

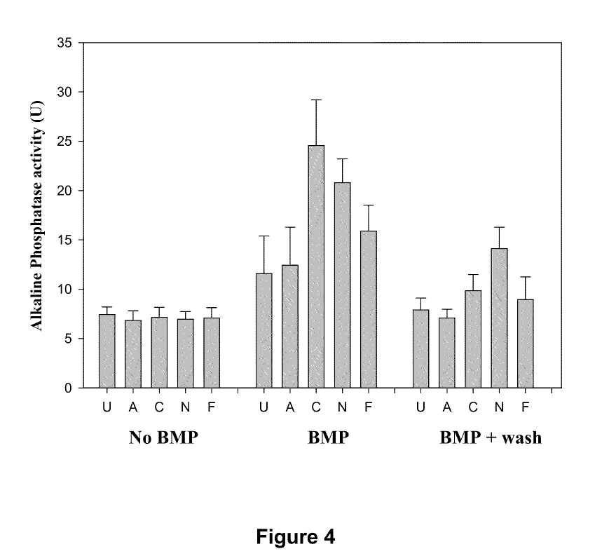

Cells cultured on Noggin or collagen had significantly greater increase in alkaline phosphatase activity with exposure to BMP than those cultured on other substrata (P<0.01) (FIG. 4).

In cultures where the BMP had been washed away prior to the seeding of cells, ALP levels were elevated compared to controls only in the Noggin coated wells (P<0.001, ANOVA, Holm Sidak post hoc test) (FIG. 4).

C2C12 cells were cultured in wells which were uncoated (U), or coated with Albumin (A), Collagen I (C), Noggin (N), or Fetuin (F), which had been incubated with buffer (No BMP), BMP (BMP), or BMP followed by a wash step (BMP+wash). Cells cultured on Collagen and Noggin showed the greatest ALP activity in the presence of BMP-2. In the cultures where BMP was washed away prior to cell seeding only the Noggin coated wells retained BMP activity.

This example demonstrates that the activity of the growth factor retained on the antagonist-GF coated surface is preserved.

### Example 6

### An In Vivo Assay to Test the Osteoinductive Activity of a Bioimplant Containing BMPs

Materials & Methods

The osteoinductive capacity of recombinant hBMP-2 protein was measured using the mouse implantation model of osteoinduction, which has been previously described (see, for example, Urist et al. Meth Enzym. 1987:146;294-312).

Test BMP samples include rhBMP-2 or rhBMP-7 samples with carriers. The carriers include BMP co-lyophilized with atelopeptide type I collagen carrier (Collagen Corp Palo Alto, Calif. (rhBMP-2), or OP-1 implants (rhBMP-7) Stryker Kalamazoo, Mich.); BMP in solution added to atelopepetide type I collagen carrier (Infuse implants (rhBMP-2), Medtronic, Minneapolis, Minn., or Collagen Corp rhBMP-7); BMP co-lyophilized with a collagen carrier and a BMP antagonist; BMP in solution added to a BMP antagonist co-lyophilized with a collagen carrier; BMP lyophilized on an alloplast (ceramic, calcium phosphate, polymer or metal) with or without an antagonist co-lyophilized to the alloplast; and BMP in solution applied to an alloplast, with or without a antagonist coating lyophilized on the alloplast.

Swiss-Webster mice (Harlan Sprague-Dawley, Indianapolis, Ind.) were anesthetized by isoflurane gas and placed on the table in a prone position. A 1 by 2 cm site was shaved in the dorsum of the lumbar spine extending over both hips. The site was prepared with 70% alcohol solution. A 10 mm skin incision was made perpendicular to the lumbar spine and muscle pouches were created in each hind quarter. Mice were implanted with 50pg of rhBMP-2 or rhBMP-7 in collagen carriers (Infuse & OP-1 respectively). The BMP implant, placed in no. 5 gelatin capsules (Torpac Inc. Fairfield, N.J.), was implanted in the muscle pouches and the wounds closed with metal clips (Poper, Long Island, N.Y.).

Animals received a BMP-2 capsule implant in one hind quarter muscle mass, with the contralateral muscle mass being implanted with the carrier alone.

The animals were sacrificed 28 days post-implantation and the hind quarters were dissected from the torso. The specimens were fixed in buffered neutral 10% formalin for a minimum of 24 hours and the amount and quality of bone induced by the BMP containing implants was determined by microCT.

The mean values for the OP-1® treated mice were significantly higher than those treated with Infuse® with regards to total volume (P=<0.001), bone volume (P=0.031 using the Mann-Whitney Rank Sum Test, MWRST), bone mineral content (P=0.023), and tissue mineral content (P=0.045 using the MWRST).

The inventors have improved the quantification of induced heterotropic bone formation in mice by using a micro-CT scanner, rather than radiographs as described in the art. The hind quarters were imaged using a microCT™ scanner (eXplore™ Locus, GE Healthcare, London, ON, Canada). Micro CT is a technique that uses x-rays to generate a series of radiographs along three planes of a specimen, which are later digitized and used to create a 3D computer model that enables the evaluation of the induced bone.

Once the 3D construct is produced the ossicle of included bone caused by the BMP implant is outlined as a region of interest (ROI). All analysis was restricted to this ROI.

This ROI, however, is not pure bone, and also includes the volume occupied by blood, muscle tissue and fat. To exclude these less dense tissues from the measurement, a threshold value of 30% of the bone standard included in each micro CT scan was used as the cut off density value, giving a measurement of the bone volume. A percentage of the bone standard is used as a threshold, rather than an absolute value in order to control for the scan to scan variability that was observed.

This method is more sensitive and provides better resolution than microradiographs and provides volume measurements compared to area measurements provided by microradiographs or histological analysis. Consequently the quantification of induced bone using microCT is more accurate than that estimated from microradiographs.

Once the microCT analysis was completed the implants are excised and embedded in paraffin. Ten micron sections are prepared and stained with hematoxylin-eosin and azure II. Hematoxylin-eosin von Kossa's staining is used to identify sites of calcification.

Results

The total induced bone was evaluated by micro CT using seven standard bone quantity and bone quality parameters (total volume of the ROI (TV) bone mineral content within the ROI (BMC), bone mineral density (BMD), bone volume (BV), tissue mineral content (TMC), tissue mineral density (TMD) and bone volume fraction (BVF).

The amount of bone produced by the BMP is indicated by the measurements for TV, BV, BMC and TMC. The quality of the bone is evaluated by the measurements of BMD, TMD and BVF.

When comparing BMP-2-containing Infuse® implants and BMP-7-containing OP-1® implants the mean values for the OP-1® treated mice were significantly higher than those treated with Infuse® with regards to total volume (P=<0.001), bone volume (P=0.031 using the Mann-Whitney Rank Sum Test, MWRST), bone mineral content (P=0.023), and tissue mineral content (P=0.045 using the MWRST).

No significant differences were found between the mean values of OP-1® and Infuse® treated mice with regards to measures of bone quality, specifically bone mineral density (P=0.600), tissue mineral density (P=0.186 using the Mann-Whitney Rank Sum Test), and bone volume fraction (P=0.550).

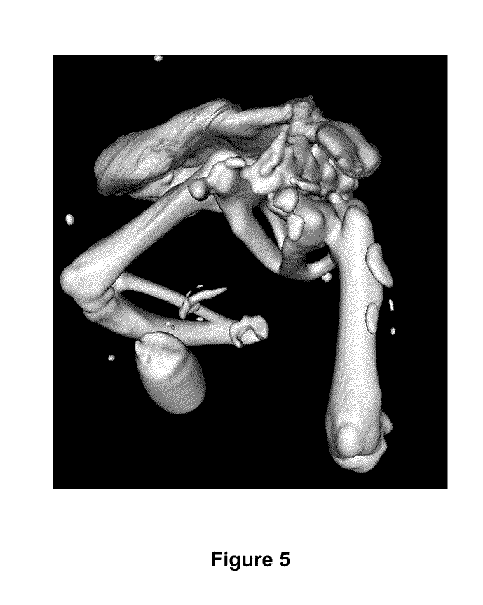

FIG. 5 shows a MicroCT™ scan of a mouse hindquarters implanted with rhBMP-7 in a collagen sponge. The image is of a 3D computer model that was generated from the series of radiographs acquired during micro CT scanning. The rhBMP-7 induced bone has been highlighted.

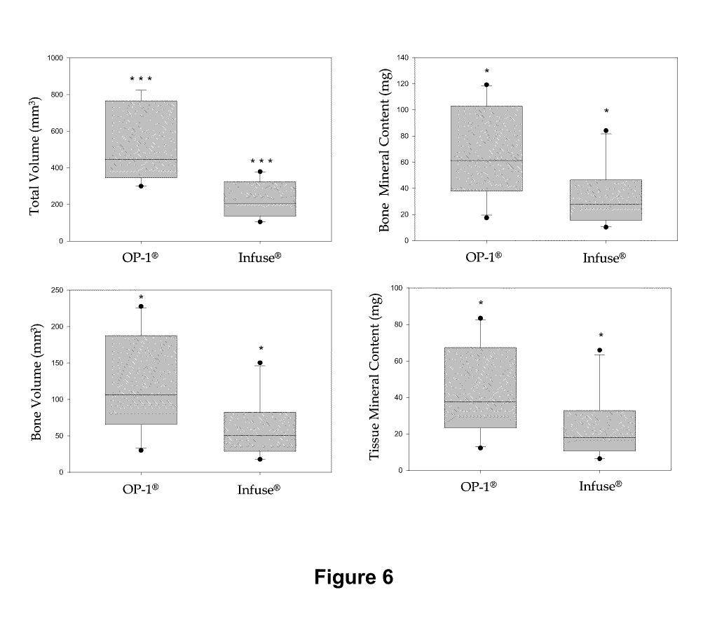

FIG. 6 shows the results of analysis of the bone formed by rhBMP-2 in solution applied to a collagen implant (Infuse) and rhBMP-7 lyophilized onto collagen (OP-1) using microCT.

This example demonstrates the effectiveness of bioimplants containing BMP combined with or without an antagonist to induce bone formation.

### Example 7

### Evaluation of a Calcium Phosphate Carrier Combined with BMP and Pluronic F127 Delivery Vehicle In Vivo

Materials & Methods

C-Graft™ (100% HA) and the Algisorb™ (a biphasic calcium phosphate, BCP), were both provided by Citagenix Inc. (Laval, Canada). These bioimplants were in granular form ranging in size from 300 to 1,000 microns.

The BMP used for this investigation was rhBMP-7 (OP-1® Stryker Biotech Inc. Hopkinton, Mass., USA).

Pluronic™ F127 (F127) was obtained from Sigma Aldrich (St. Louis Mo.). Stock F127 was prepared as follows: 100 ml of MilliQ™ water was chilled to 4° C. 33 g of F127 was slowly added over a period of several hours while stirring at 4° C. Once all the F127 was dissolved the stirrer bar was removed and the F127 stock solution was autoclaved to sterilize.

Experimental Design

Twenty-five skeletally mature New Zealand White male rabbits (Charles River Laboratories, Montreal, QC, Canada) weighing 3.5 to 4.0 kg were randomly divided into groups of 5 animals each. Two 15 mm diameter critically sized defects were made in the parietal bones of each rabbit. All of the animals were sacrificed at 6 weeks.

Surgical Protocol

The surgical procedures for this investigation were performed according to recognized techniques approved by the University of Toronto, Animal Care Ethics Committee (NO. 20005030). Each animal was pre-medicated according to their weight with a composite of acepramazine (1 mg/kg), ketamine (35 mg/kg), and zylazine (2 mg/kg). General anesthesia was induced using intravenous sodium thiopental (20 mg/kg). After induction, a 3 mm uncuffed endotracheal tube was used for intubation. Anesthesia was maintained with 1:1.5% insoflurane and oxygen composite using mechanical ventilation. The animals were monitored using pulse oxymetry. Respiration rate of the animal was set at 20 breaths per minute with a tidal volume of 10 ml/kg.

An incision was made along the midline of the scalp from a point midway between the base of the ears to approximately 5 cm anteriorly through full-thickness skin. Sharp subperiosteal dissection reflected the pericranium from the outer table of the cranial vault exposing the parietal bones. An electric drill with a 702 fissure bur under copious saline irrigation was used to create bilateral full-thickness calvarial defects. The defects were ovoid in shape measuring 15 mm by 13 to 15 mm. A surgical template was used to define the defect margins. Two defects were created, one on each side of the midline. The bioimplants were placed directly to fill the defects. Care was taken to prevent displacement of the test materials into other defect (cross contamination). The pericranium and skin were closed with resorbable sutures.

Test Groups

Group 1: Autogenous vs. Unfilled

In Group 1 (n=5), each animal had one defect left unfilled to serve as the control. These defects were allowed to heal spontaneously. The contralateral defects were filled with morcelized autogenous bone, which was the bone removed from the calvaria.

Group 2: C-Graft vs. C-Graft+Pluronic

Animals in Group 2 (n=5) had one defect filled with 0.4 g of C-Graft mixed with 0.56 ml of Pluronic. The contralateral defect was filled with 0.4 g of C-Graft mixed with blood. Clotted blood increases the adhesion between granules to bone and this procedure has been used in general practice 18.

Group 3: C-Graft+Pluronic vs. C-Graft+Pluronic+BMP

For animals in Group 3 (n=5), one defect was filled with 0.4 g of C-Graft mixed with 0.56 ml of Pluronic. The contralateral defect was filled with 0.4 g of C-Graft mixed with 0.56 ml of Pluronic and 50 mg of OP-1.

Group 4: Algisorb vs. Algisorb+Pluronic

In this group (n=5), one defect was filled with 0.4 g of Algisorb, and the contralateral defect was filled with 0.4 g Algisorb with 0.58 ml of Pluronic. Algisorb was dipped in blood to form clots.

Group 5: Algisorb+BMP vs. Algisorb+BMP+Pluronic

In Group 5 (n=5), one defect was filled with 0.4 g Algisorb with 50 mg of OP-1®. The contralateral defect was filled with a mixture of 0.4 g Algisorb with 50 mg of OP-1® and 0.56 ml of Pluronic.

Histology

All animals were sacrificed at 6 weeks. The cranial vault was carefully removed from each animal. The calvaria specimens were placed in 10% neutral buffered formalin for 72 hours, decalcified in formic acid, and embedded in paraffin. Multiple 6 μm sections were cut from the middle of each specimen and stained with hematoxylin-eosin (H&E) for quantification of the amount of bone regeneration under light microscropy.

Histomorphometry

Histomorphometric analysis was performed first by viewing the H&E stained sections under a light microscope (Leitz, Wetzlar, Germany) at ×1.25 magnification. The sagittal suture was used as a landmark to identify the site of defects. Multiple serial pictures of specimens were captured using an RT Color digital camera (Diagnostic Instruments Inc., Sterling Heights, Mich.) attached to the microscope and displayed on the computer monitor. The serial pictures were then merged into one image using Adobe Photoshop Element 2.0 software. The merged images were calibrated and quantified.

The areas of total defect, new bone, bone marrow space, residual biomaterial, and soft tissue were measured from the merged images using Image Pro® Plus 4.3 software (Media Cybernetics, Carlsbad, Calif.). These measurements were made on five histology slides for each animal. Measurement data were exported to Microsoft Excel once compiled. Measurements were expressed as a percentage of the total defect area.

Statistical Analysis

Histomorphometric results were analyzed using SigmaStat® 3.0 (Systat Inc. Point Richmond, Calif.) statistical software. Comparison of contralateral treatments within the same group was done by paired T-tests. One-way ANOVA or Two-way ANOVA was performed to evaluate for statistical significance between the various groups and the SNK post hoc test was used to determine which groups were significantly different. Statistical significance was established at P<0.05.

Results

All animals survived the surgical procedure and were available for analysis. Gross examination at necropsy showed no signs of inflammatory reaction in any of the defects.

Histological Evaluation

Unfilled Defects

At 6 week, fibrous tissue filled most of the defect. Healing of the defects was mainly by scar formation. Bony in-growth was visible at margins of the defects. Some defects had a few bony islands close to the dural lining.

Autogenous Bone

Histological analysis demonstrated complete union across all defects making the defect margins indistinguishable. These defects were filled with the implanted autogenous bone and newly formed woven bone. New bone contained marrow spaces which were highly cellular. Large numbers of red blood cells (RBCs) were visible, indicating vascularization. The pericranial contour appeared convex due to increased bone height.

C-Graft-Filled Defects

Histological examination revealed complete bony union across all defects. Integration of new bone to the presurgical bone occurred at the defect margins. New bone formation was observed, which was distinguished by its more intense staining. The height of regenerated bone was slightly thinner at the centre of the defect, mostly on the brain side. C-Graft™ granules appeared complete with little sign of degradation and resorption. Little or no bony in-growth was observed within the granules.

C-Graft+Pluronic

Defects filled with G-Graft™+Pluronic™ appeared indistinguishable from the defects filled with C-Graft™ by histological evaluation.

C-Graft+Pluronic+BMP

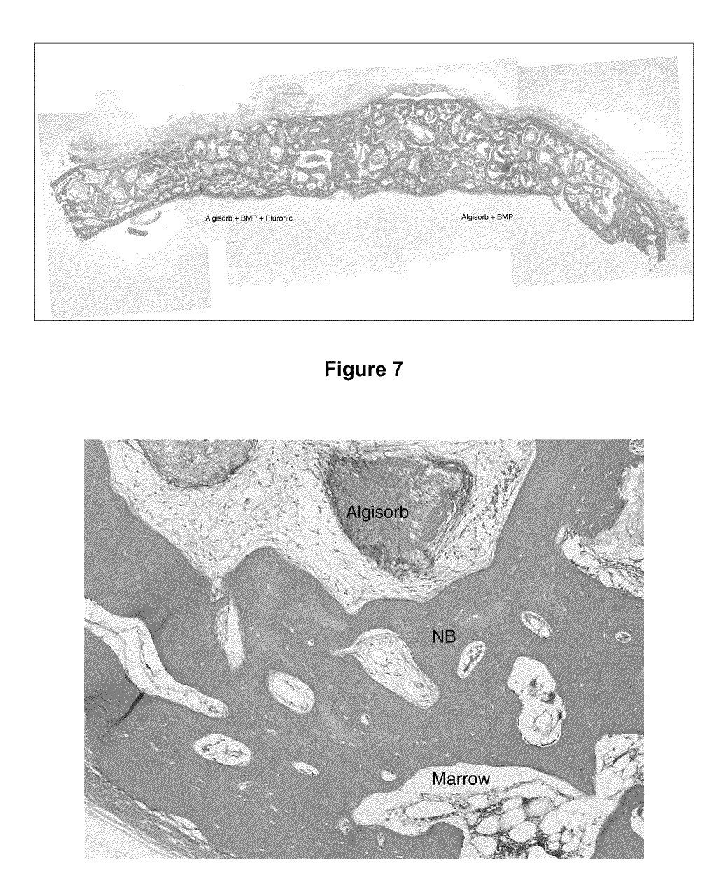

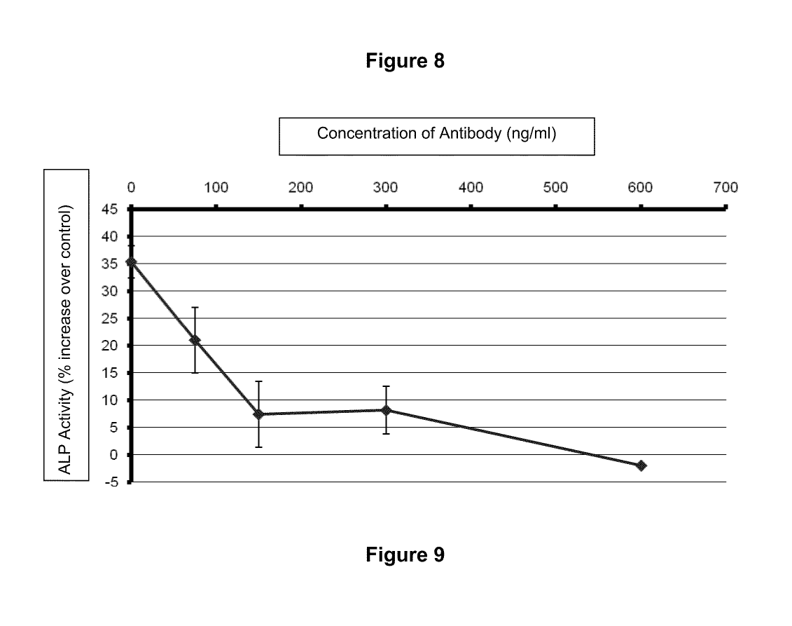

Histologically, C-Graft™+Pluronic+BMP-filled defects demonstrated bone growth across the entire defect (FIG. 7). The amount of new bone and marrow space was greater than that of C-Graft™ filled or C-Graft™+Pluronic filled defects (FIG. 8). There was little evidence of C-Graft™ degradation, as the granules appeared complete and had not lost their original morphology.

Algisorb

Histological examination revealed that Algisorb was able to conduct new bone formation. Integration of new bone to the presurgical bone occurred at the defect margins. There was minimal amount of bony in-growth into the Algisorb granules. Algisorb granules demonstrated some degradation as its granules appeared less compact and had voids within them and bone was observed within the body of the granules.

Algisorb+Pluronic

Defects filled with Algisorb appeared indistinguishable from the defects filled with Algisorb+Pluronic by histological evaluation.

Algisorb+BMP

Histologically, defects had complete bony union. A desirable thickness was achieved for all the defects, making them comparable to the defects filled with autogenous bones. Defects filled with Algisorb+BMP had greater amount of new bone formation and marrow spaces than defects treated without Algisorb alone or Algisorb+Pluronic. There were increased numbers of voids around the Algisorb granules, which might have been a result of rapid degradation of the β-TCP.

Algisorb+BMP+Pluronic

Defects filled with Algisorb+BMP+Pluronic were indistinguishable histologically from Algisorb+BMP-filled defects (FIG. 7, 8).

Histomorphometric Analysis

Histomorphometry results are summarized in Tables 1 and 2. After 6 weeks, the autogenous-filled defects demonstrated a significantly higher volume of bone and marrow (reparative tissue) than the control (unfilled defects) (P<0.001).

In the groups treated using C-Graft (groups 2 and 3), C-Graft-filled and C-Graft+Pluronic-filled defects demonstrated similar amounts of new bone and marrow. The defects reconstructed using C-Graft+Pluronic+BMP contained significantly greater volume of reparative tissue compared to defects filled with C-Graft+Pluronic alone (P=0.007).

In the groups with Algisorb (groups 4 and 5), Algisorb-filled and Algisorb+Pluronic-filled defects demonstrated similar volume of new bone and marrow. The amount of reparative tissue was significantly higher when BMP was added to Algisorb. Algisorb+BMP-filled and Algisorb+BMP+Pluronic-filled defects demonstrated desirable volume of reparative tissue, almost reaching the amount formed by defect filled with autogenous bones. While no differences were seen in the amount of reparative tissue when Pluronic was added to Algisorb, there was slightly more reparative tissue in grafts containing Algisorb+BMP compared to those containing Algisorb+BMP+Pluronic (P=0.048).

By ANOVA, addition of Pluronic to C-Graft™ and Algisorb did not demonstrate statistical significance on the percentage of reparative tissue regenerated (P=0.355 and P=0.876, respectively). However, the addition of BMP to C-Graft™ and Algisorb considerably increased the amount of new bone compared to void-filled defects (P=0.003 and P=0.006, respectively). Within the limits of this investigation, we were unable to demonstrate significant difference in the healing that resulted from using C-Graft™ and Algisorb.

FIG. 7 provides a panoramic picture of the bilateral calvarial defects created by merging sectional photomicrographs from Algisorb+BMP+Pluronic versus Algisorb+BM filled defects at 6 weeks (Group 5).

FIG. 8 shows a low power photomicrograph of Algisorb+BMP+Pluronic filled defect at 6 weeks. Large amounts of bone marrow are also present.

TABLE 1
Percentage of defect filled with reparative tissue at 6 weeks.

| | | Reparative | |
|---|---|---|---|
| | | Tissue | |
| Group | Treatment | (%) | P^{a} |
| Group 1 | Unfilled | 28.9 ± 3.8 | <0.001 |
| | Autogenous | 82.3 ± 3.5 | |
| Group 2 | C-Graft | 35.2 ± 10.3 | 0.355 |
| | C-Graft + Pluronic | 42.5 ± 14.5 | |
| Group 3 | C-Graft + Pluronic | 36.4 ± 12.7 | 0.007 |
| | C-Graft + Pluronic + BMP | 65.5 ± 15.9 | |
| Group 4 | Algisorb | 34.1 ± 27.3 | 0.972 |
| | Algisorb + Pluronic | 34.3 ± 31.5 | |
| Group 5 | Algisorb + BMP | 75.8 ± 15.5 | 0.048 |
| | Algisorb + BMP + Pluronic | 69.5 ± 19.2 | |

^{a}The P value was calculated using the paired t-test.

The results represent the percentage of the defect filled with reparative tissue (bone and marrow), and are presented as mean±SD.

TABLE 2
Percentage of defect filled with residual biomaterial at 6 weeks.

| | | Residual | |
|---|---|---|---|
| | | Biomaterial | |
| Group | Treatment | (%) | P^{a} |
| Group 2 | C-Graft | 30.1 ± 5.1 | 0.745 |
| | C-Graft + Pluronic | 28.9 ± 9.3 | |
| Group 3 | C-Graft + Pluronic | 22.5 ± 8.6 | 0.200 |
| | C-Graft + Pluronic + BMP | 17.2 ± 4.9 | |
| Group 4 | Algisorb | 33.8 ± 14.0 | 0.822 |
| | Algisorb + Pluronic | 32.6 ± 17.9 | |
| Group 5 | Algisorb + BMP | 14.6 ± 8.8 | 0.125 |
| | Algisorb + BMP + Pluronic | 19.3 ± 10.6 | |

^{a}The P value was calculated using the paired t-test.

Results represent the percentage of the defect filled with residual biomaterial, and are presented as mean±SD.

This example demonstrates the effectiveness of a bioimplant, composed of a biomaterial carrier combined with a GF and a delivery vehicle to promote bone healing.

### Example 8

### Evaluation of Bioimplant Composed of a Carrier with Immobilized Antagonist Combined with Growth Factor and Delivery Vehicle to Promote Bone Repair In Vivo

Materials and Methods

Carrier

Sterile 100% Hydroxyapatite (HAp) and biphasic calcium phosphate (BCP), were both provided by Citagenix Inc. (Laval, Canada). These bioimplants were in granular form ranging in size from 300 to 1,000 microns.

Antagonist

Recombinant mouse Noggin (NGN) is available from RnD Systems (Minneapolis, Minn.). The mouse Noggin was re-suspended in 0.1 M HCl and sterilized by filtration through 0.2 μm filters.

Growth Factor

Sterile recombinant human BMP-2 (rhBMP-2) used for this investigation was obtained from the Infuse™ bone graft kit (Medtronic Minneapolis, Minn.). The rhBMP-2 was provided as a lyophilized powder which was reconstituted in 1 ml sterile water. Upon reconstitution the growth factor solution contained 1.5 mg rhBMP-2, 5 mg sucrose, 25 mg glycine, 3.7 mg L-glutamine, 0.1 mg sodium chloride and 0.1 mg polysorbate 80 per ml.

Delivery System

Pluronic™ F127 (F127) was obtained from Sigma Aldrich (St. Louis Mo.). Stock F127 was prepared as follows. 100 ml of MilliQ™ water was chilled to 4° C. 33 g of F127 was slowly added over a period of several hours while stirring at 4° C. Once all the F127 was dissolved the stirrer bar was removed and the F127 stock solution was autoclaved to sterilize.

Preparation of the Bioimplants

Noggin (NGN) was incubated with the HAp and BCP granules at a ratio of 100 to 1000 μg NGN per gram of carrier at 4° C. for 30 minutes, under gentle agitation. The carrier-antagonist (C-A) preparation was then lyophilized. Following lyophilization the C-A was sterilized by exposure to chloroform vapor.

At the time of surgery the C-A preparations were mixed with or without rhBMP-2 (10 to 200 μg) and or F127 (2:1 v/v carrier:F127).

Surgical Protocol

Bilateral critical sized 15mm calvarial defects were made in the parietal bones of rabbits as described as in Example 7. The bioimplants were placed directly into the defects filling them. Care was taken to prevent displacement of the test materials into other defect (cross contamination). The pericranium and skin were closed with resorbable sutures

MicroCT Analysis

All animals were sacrificed at 6 weeks. The cranial vault was carefully removed from each animal and was placed in 10% neutral buffered formalin for 72 hours. Samples were then scanned by microCT™ as described in Example 6. Eppendorf tubes containing the carriers were also scanned.

Following reconstruction and identification of the regions of interest the analysis was performed twice using two threshold values. The first threshold was set at 60% of the value obtained for the carriers. Voxels with a greyscale higher than this value were considered to be carrier. The output for bone volume and BVF were thus considered to represent carrier volume (CV) and carrier volume fraction (CVF). The second analysis was done with a lower threshold which represents 20% of the bone standard. The amount of bone was determined by subtracting the CV obtained at the higher threshold from the BV obtained at the lower threshold.

Histology & Histomorphometry

The samples were then prepared and analysed by histology and histomorphometry as described in example 7.

Statistical Analysis

Statistical analysis was performed as described in Example 7.

This example demonstrates that an antagonist coated carrier can be associated with a growth factor as a delivery vehicle and that such a bioimplant can be used to promote bone healing.

### Example 9

### Demonstration of the Use of Antibodies that are Antagonists to BMP Activity for Retention of BMP Activity Once Immobilized to a Surface

As an example of how to screen for antagonist antibodies that enhance the retention of BMP activity on a surface we carried out the following experiments.

a) Testing for Antagonist Activity

Materials & Methods

Purified polyclonal rabbit anti-human BMP-2 antibodies were purchased from Cell Sciences (Canton, Mass., USA, Cat #PA0025).

To determine whether the antibody was an antagonist, 45 ng of carrier free recombinant human BMP-2 (RnD Systems, Cat #355-BM/CF) were incubated with 0, 75, 150, 300 and 600 ng of antibody in 0.5 ml of alpha MEM+15% FBS. These concentrations represent approximate molar ratios of BMP:AB of approximately 3:1, 3:2, 3:4 and 3:8 (assuming approximate molecular weights for rhBMP-2 and the antibody of 32 Kd and 150 Kd respectively). After incubation for an hour the medium containing the BMP and antibody was added to wells of previously seeded C2C12 cells containing 0.5 mls of aMEM+15% FBS (prepared as described in Example 1).

After 2 days the cultures were terminated, the cell layers lysed and the lysates assayed for alkaline phosphatase (ALP) and protein (PTN) as described in Example 1. More specifically, media containing different concentrations of an anti-BMP-2 antibody with or without 45 ng rhBMP-2 was pre-incubated prior to addition to wells containing C2C12 cells. Following 2 days of culture the cells were then assayed for ALP activity and the increase in ALP activity with the addition of BMP was plotted against the antibody concentration.

The amount of BMP activity was determined by converting the ALP results to percent increase above control (ALP from wells with the matching concentration of antibody but no BMP).

As can be seen from the graph in FIG. 9, the increase in ALP activity with the addition is reduced with increasing concentration of antibody.

Results

Addition of BMP-2 with no antibody resulted in a significant increase in ALP compared to control wells that had only aMEM+15% FBS added. In wells with BMP plus antibody the increase in ALP activity over controls (treated with antibody at the same concentration but no BMP) was seen to decline. At the highest antibody concentration tested the ALP activity was not significantly different from control. The results are shown in FIG. 9.

b) Testing for Retention of BMP Activity by Immobilized Antibody

Materials & Methods

Wells of 24 well tissue culture plates were coated with 100 ng of AB-PA0025, BSA or left untreated (Control). The proteins were prepared in 100 μl PBS and left to incubate in a laminar flow hood for 2 hours. The coating solution was then removed and the wells rinsed with PBS. After rinsing the plates were sterilized under UV light for 30 minutes.

Following sterilization, 0.5 ml of aMEM+15%/FBS with or without carrier free rhBMP-2 (90 ng) was added to each well and incubated for 1 hour. Following this some wells had the medium aspirated and fresh 0.5 ml aMEM+15% FBS containing no rhBMP-2 was added. Finally to all wells 0.5 ml of C2C12 cells in aMEM+15% FBS was added (final concentration 0.5×10⁵ cells/ml). After 2 days the cultures were then assayed for alkaline phosphatase activity (ALP) and protein (PTN) content as described in Example 1.

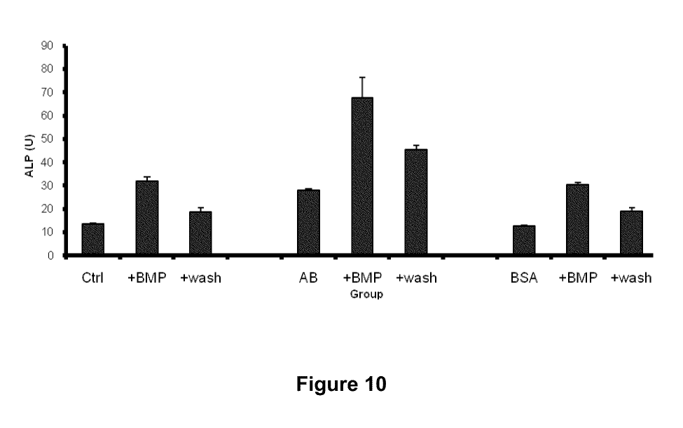

The results of this example are illustrated in FIG. 10, which shows C2C12 cells were cultured in wells, which were uncoated (Ctrl), or coated with antibody (AB) or Albumin (BSA), which had been incubated with buffer (1^{st} bar of set) or BMP (BMP, 2^{nd} bar of set), or BMP followed by a wash step (+wash, 3^{rd} bar of each set). Cells cultured on all surfaces had increased ALP activity if BMP was present in the medium. If the medium containing BMP had been removed (+wash) then the wells pretreated with antibody had the highest ALP activity, which demonstrated the ability of the antibody to act to improve retention of BMP activity.

Results

Seeding of C2C12 cells into media containing BMP-2 increased ALP activity significantly in all groups no matter what the plates had been coated with. However, if the media containing the BMP was removed prior to cell seeding and replaced with media containing no rhBMP-2 the wells that had been incubated with antibody to allow it to immobilize on the surface showed the greatest ALP activity. Results were as shown in FIG. 10.

This example demonstrates that a surface coated with an antibody antagonist can retain GF activity on the coated surface.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1-44. (canceled)

45. A method for adapting a substrate to deliver growth factor activity at a localized site, the method comprising immobilizing an antagonist of the growth factor on the substrate and binding the growth factor to the antagonist.

46. The method of claim 45, wherein the substrate comprises a surface of a bioimplant.

47. The method of claim 45, wherein the substrate comprises a carrier for the antagonist.

48. The method of claim 45, wherein the growth factor is releasably bound to the antagonist or wherein the growth factor retains its activity when bound to the antagonist.

49. The method of claim 45, wherein the growth factor is a member of the transforming growth factor beta superfamily and the antagonist is an antagonist of the transforming growth factor beta superfamily member.

50. The method of claim 49, wherein the growth factor is a transforming growth factor beta (TGF(β) and the antagonist is a TGFβ antagonist.

51. The method of claim 50, wherein the transforming growth factor beta is TGFβ or TGFβ2.

52. The method of claim 50, wherein the antagonist is latency associated peptide (LAP), TGFβsRII, or an antibody having a binding region that is complementary to a region of the growth factor.

53. The method of claim 45, wherein the growth factor is a bone morphogenetic protein (BMP) and the antagonist is a BMP antagonist.

54. The method of claim 53, wherein the bone morphogenetic protein is rhBMP-2 or rhBMP-7.

55. The method of claim 53, wherein the antagonist is a Noggin, chordin, gremlin, sclerostin, or an antibody having a binding region that is complementary to a region of the growth factor.

56. The method of claim 45, wherein the growth factor is an insulin like growth factor (IGF) and the antagonist is an insulin like growth factor binding protein.

57. The method of claim 56, wherein the insulin like growth factor is IGF-1 or IGF-2.

58. The method of claim 56, wherein the antagonist is an IGF binding protein or an antibody having a binding region that is complementary to a region of the growth factor.

59. The method of claim 45, wherein the antagonist is an antibody having a binding region that is complementary to a region of the growth factor or is a genetically engineered fragment of the growth factor receptor.

60. The method of claim 45, wherein the antagonist is immobilized onto a carrier and wherein the carrier is: a synthetic and/or natural calcium phosphate; a synthetic and/or natural polymer; a protein, such as collagen type I; or a metal.

61. The method of claim 45, wherein the antagonist is genetically modified to include an amino acid sequence which promotes immobilization onto the surface.

62. A bioimplant adapted to deliver an exogenous growth factor at a site of implantation, the bioimplant comprising a surface having immobilized thereon an antagonist of the growth factor and wherein the growth factor is bound to the antagonist.

63. The bioimplant of claim 62, wherein the growth factor is a member of the transforming growth factor beta superfamily and the antagonist is an antagonist of the transforming growth factor beta superfamily member.

64. The bioimplant of claim 63, wherein the growth factor is a transforming growth factor beta (TGFβ) and the antagonist is a TGFβ antagonist.

65. The bioimplant of claim 64, wherein the transforming growth factor beta is TGFβ1 or TGFβ2.

66. The bioimplant of claim 62, wherein the growth factor is a bone morphogenetic protein (BMP) and the antagonist is a BMP antagonist.

67. The bioimplant of claim 66, wherein the bone morphogenetic protein is rhBMP-2 or rhBMP-7.

68. The bioimplant of claim 62, wherein the growth factor is an insulin like growth factor (IGF) and the antagonist is an insulin like growth factor binding protein.

69. The bioimplant of claim 68, wherein the insulin like growth factor is IGF-1 or IGF-2.

70. The bioimplant of claim 62, wherein the antagonist is: (i) an antibody having a binding region that is complementary to a region of the growth factor; or (ii) genetically modified to include an amino acid sequence which promotes immobilization onto the surface.

71. The bioimplant of claim 62, wherein the bioimplant comprises a carrier such as: a synthetic and/or natural calcium phosphate; a synthetic and/or natural polymer; a protein; or a metal.

72. A method of delivering growth factor activity to a desired location in an organism comprising:
providing a substrate;
immobilizing an antagonist of said growth factor on said substrate;
binding the growth factor to the antagonist; and,
implanting the substrate at the desired location.

73. The method of claim 72, wherein the substrate comprises a surface of a bioimplant.

74. The method of claim 72, wherein said substrate further comprises a carrier to which the antagonist is immobilized.

75. The method of claim 72, wherein the growth factor is: a member of the transforming growth factor beta superfamily; an insulin-like growth factor (IGF); or bone morphogenetic protein (BMP).

76. The method of claim 72, wherein the antagonist is: (i) an antibody having a binding region that is complementary to a region of the growth factor; or (ii) genetically modified to include an amino acid sequence which promotes immobilization onto the surface.
